(19) United States
(12) Patent Application Publication
(10) Pub. No.: US 2026/0174742 A1
(43) Pub. Date: Jun. 25, 2026

(54) INHIBITORS OF CYCLIC ADP RIBOSE HYDROLASE AND METHODS OF USE THEREOF

(71) Applicant: Flagship Pioneering Innovations VI, LLC, Cambridge, MA (US)

(72) Inventors: Benito Munoz, Newtonville, MA (US); Hairuo Peng, Needham, MA (US)

(21) Appl. No.: 19/128,518

(22) PCT Filed: Nov. 9, 2023
(86) PCT No.: PCT/US2023/079254

§ 371 Date: May 8, 2025

Related U.S. Application Data
(60) Provisional application No. 63/424,232, filed on Nov. 10, 2022.

(51) Int. Cl. A61K 31/4439

(52) U.S. Cl. CPC A61K 31/4439

(57) ABSTRACT

The disclosure provides compounds, in part, compounds of Formula I or Formula II and their use in treating medical diseases or disorders, such as neurodegenerative diseases, e.g., Parkinson's disease. Pharmaceutical compositions and methods of making compounds of the disclosure are provided. The compounds are contemplated to be modulators, e.g., inhibitors, of cyclic ADP ribose hydrolase (CD38).

### CROSS-REFERENCE TO RELATED APPLICATIONS

This application claims the benefit of, and priority to, U.S. Ser. No. 63/424,232 filed on Nov. 10, 2022; the content of which is incorporated herein by reference in its entirety.

### BACKGROUND

The glycoprotein CD38 (cluster of differentiation 38), also known as cyclic ADP ribose hydrolase, is a multifunctional enzyme that, for example, catalyzes the degradation of nicotinamide adenine dinucleotide (NAD+) to adenosine diphosphate ribose (ADP ribose or ADPR). CD38 can also serve as a cyclase converting NAD+ to cyclic ADP-ribose (cADPR). Its NADase activity is more predominant than its function as an ADP-rybosyl-cyclase, being 100-fold less efficient in cyclizing NAD+ to cADPR than in hydrolzing NAD+ to ADP ribose. CD38 can also hydrolyze nicotinamide adenine dinucleotide phosphate (NADP) to nicotinic acid adenine dinucleotide phosphate (NAADP), both of which are derived from NAD+. Thus. CD38 is thought to be a significant consumer of NAD+ and a regulator of NAD+ levels.

Dysregulation of NAD+ levels is associated with a variety of disease states. For example, disease states associated with NAD+ depletion and disregulation in NAD+ related metabolites include, but are not limited to, e.g., obesity, diabetes, cancer, heart disease, asthma, and inflammation. Therefore, returning NAD+ to its normal level in disease states that consume significant amounts of NAD+ may result in therapeutic benefits.

For example, reduced NAD+ levels are associated with aging, and this age-related dysfunction may contribute to increased neurogeneration and the onset of neurodegenerative disorders. It is contemplated that expression of CD38, the enzyme responsible for NAD+ degradation, increases as a consequence of aging, thus providing a plausible rational for decreased NAD+ levels as a consequence of aging. As such, one approach to modulating cellular NAD+ levels may be inhibiting enzymes that consume NAD+, for example, CD38.

Inhibition of CD38 enzyme activity, which in turn modulates NAD+ tissue levels, presents a useful approach for the treatment of diseases associated with increased CD38 expression and/or decreased cellular NAD+ levels. Therefore, there is an ongoing need for small molecule inhibitors of CD38 in treating diseases or conditions responsive to modulation, e.g., inhibition, of cellular levels of NAD+.

### SUMMARY

The disclosure is directed, at least in part, to compounds that modulate, e.g., inhibit, the expression and/or activity of CD38. Also disclosed herein are pharmaceutical compositions comprising at least one disclosed compound and a pharmaceutically acceptable excipient.

For example, disclosed herein is a compound represented by Formula I:

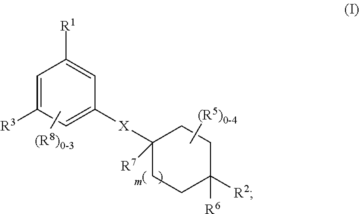

or a pharmaceutically acceptable salt and/or a stereoisomer thereof, wherein:
   X is —C(O)—NR⁴— or —S(O)₂—NR⁴—;
   R¹ is 5-6 membered monocyclic heteroaryl or 8-10 membered bicyclic heteroaryl; wherein R¹ may optionally be substituted by one or more substituents each independently selected from R¹¹;
   R² is selected from the group consisting of —C₁₋₆alkoxy, —C₁₋₆alkyl, halogen, hydroxyl, and hydrogen; wherein —C₁₋₆alkoxy and —C₁₋₆alkyl may optionally be substituted by one or more substituents each independently selected from R²²;
   R³ is selected from the group consisting of —C₁₋₆alkyl, —C₁₋₆alkoxy, 5-6 membered heteroaryl, 4-7 membered heterocyclyl, and halogen; wherein —C₁₋₆alkyl, —C₁₋₆alkoxy, 5-6 membered heteroaryl, and 4-7 membered heterocyclyl may optionally be substituted by one or more substituents each independently selected from R³³;
   R⁴ is selected from the group consisting of hydrogen and —C₁₋₃alkyl optionally substituted by one or more halogens;
   R⁵ and R⁸ are independently selected for each occurrence from the group consisting of hydrogen, deuterium, halogen, hydroxyl, —C₁₋₆alkyl, —C₁₋₆alkoxy, —CN, —NR^{a}R^{b}, —C(O)—NR^{a}R^{b}, and —NR^{a}—C(O)—R^{b};
   R⁶ and R⁷ are each independently selected from the group consisting of hydrogen and —C₁₋₃alkyl optionally substituted by one or more halogens;
   R¹¹, R²² and R³³ are independently selected for each occurrence from the group consisting of halogen, hydroxyl, —C₁₋₆alkyl, —C₁₋₆alkoxy, —C₃₋₆cycloalkyl, phenyl, —CN, —CF₃—NR^{a}R^{b}, —C(O)—NR^{a}R^{b}, —NR^{a}—C(O)—R^{b}, and deuterium; wherein —C₁₋₆alkyl and —C₁₋₆alkoxy may optionally be substituted by one or more substituents each independently selected from hydroxyl, halogen, and —C₁₋₃alkoxy;
   R^{a} and R^{b} are independently selected for each occurrence from the group consisting of hydrogen and —C₁₋₃alkyl optionally substituted by one or more halogens; and
   m is 0, 1 or 2.

Also disclosed herein is a compound represented by Formula II:

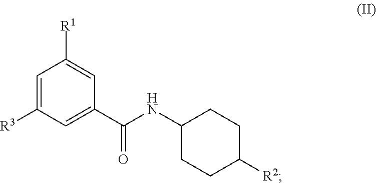

or a pharmaceutically acceptable salt and/or a stereoisomer thereof, wherein:
   R¹ is 5-6 membered heteroaryl containing at least one ring nitrogen; wherein R¹ may optionally be substituted by one, two or three substituents each independently selected from the group consisting of halogen, hydroxyl, —NH₂, —C₁₋₃alkyl, —C₁₋₃alkyl-OH, and —C₁₋₃alkoxy;
   R² is —C₁₋₆alkoxy optionally substituted by one or more substituents each independently selected from the group consisting of halogen, hydroxyl, —C₁₋₃alkyl and —C₁₋₃alkoxy; and
   R³ is selected from the group consisting of —C₁₋₆alkyl, 5-6 membered heteroaryl and 4-6 membered heterocyclyl; wherein 5-6 membered heteroaryl and 4-6 membered heterocyclyl may optionally be substituted by halogen, hydroxyl, or —C₁₋₃alkyl; and wherein —C₁₋₃alkyl and —C₁₋₃alkoxy may optionally be substituted by one or more groups selected from hydroxyl, halogen, and —C₁₋₃alkoxy.

Also disclosed herein is a method of treating a disease (e.g., a neurodegenerative disease) that benefits from inhibition of CD38 and/or an increase in NAD+ in a patient in need thereof, comprising administering to the patient an effective amount of a disclosed CD38 inhibitor. In some embodiments, the neurodegenerative disease is, for example, Parkinson's disease, Alzheimer's disease, or Huntington's disease.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₁-C₅alkenyl, C₂-C₆alkenyl, and C₃-C₄alkenyl, respectively. Exemplary alkenyl groups include, but are not limited to, vinyl, allyl, butenyl, pentenyl, etc.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁-C₅alkoxy, C₁-C₆alkoxy, and C₂-C₆alkoxy, respectively. Exemplary alkoxy groups include, but are not limited to methoxy, ethoxy, isopropoxy, etc.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Examples of representative substituted aryls include the following

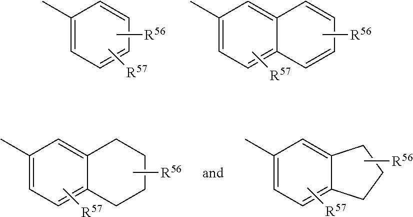

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹ NR⁵⁵SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S, R⁶⁰ and R⁶¹ are each independently hydrogen. C₁-C₈ alkyl, C₁-C₄haloalkyl, C₃-C₁₀ cycloalkyl, 4-10 membered heterocyclyl. C₆-C₁₀ aryl, substituted C₆-C₁₀ aryl, 5-10 membered heteroaryl, or substituted 5-10 membered heteroaryl.

The term “carbonyl” as used herein refers to the radical —C(O)—.

The term “cyano” as used herein refers to the radical —CN.

The terms “cycloalkyl” or a “carbocyclic group” as used herein refers to a saturated or partially unsaturated hydrocarbon group of, for example, 3-6, or 4-6 carbons, referred to herein as C₃-C₁₀cycloalkyl, C₃₋₆cycloalkyl or C₄₋₆cycloalkyl, respectively. Exemplary cycloalkyl groups include, but are not limited to, cyclohexyl, cyclopentyl, cyclopentenyl, cyclobutyl or cyclopropyl.

The terms “halo” or “halogen” as used herein refer to F, Cl, Br, or I.

The terms “haloalkyl” as used herein refers to an alkyl radical in which the alkyl group is substituted with one or more halogens. Typical haloalkyl groups include, but are not limited to, trifluoromethyl (i.e. CF₃), difluoromethyl, fluoromethyl, chloromethyl, dichloromethyl, dibromoethyl, tribromomethyl, tetrafluoroethyl, and the like. Exemplary haloalkyl groups include, but are not limited to, straight or branched hydrocarbons of 1-6, 1-4, or 1-3 carbon atoms substituted with a halogen (i.e. Cl, F, Br and I), referred to herein as C₁₋₆haloalkyl, C₁₋₄ haloalkyl, and C₁₋₃haloalkyl, respectively.

The term “hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

The terms “heteroaryl” or “heteroaromatic group” as used herein refers to an aromatic 5-10 membered ring system containing one or more heteroatoms, for example one to three heteroatoms, such as nitrogen, oxygen, and sulfur. The term may also be used to refer to a 5-7 membered monocyclic heteroaryl or an 8-10 membered bicyclic heteroaryl. Where possible, said heteroaryl ring may be linked to the adjacent radical though carbon or nitrogen. Examples of heteroaryl rings include but are not limited to furan, thiophene, pyrrole, pyrrolopyridine, indole, thiazole, oxazole, isothiazole, isoxazole, imidazole, benzoimidazole, imidazopyridine, pyrazole, triazole, pyridine or pyrimidine, etc.

The terms “heterocyclyl,” “heterocycle,” or “heterocyclic group” are art-recognized and refer to saturated or partially unsaturated 4-10 membered ring structures, whose ring structures include one to three heteroatoms, such as nitrogen, oxygen, and sulfur. Where possible, heterocyclyl rings may be linked to the adjacent radical through carbon or nitrogen. The term may also be used to refer to 4-10 membered saturated or partially unsaturated ring structures that are bridged, fused or spirocyclic ring structures, whose ring structures include one to three heteroatoms, such as nitrogen, oxygen, and sulfur. Examples of heterocyclyl groups include, but are not limited to, pyrrolidine, piperidine, morpholine, thiomorpholine, piperazine, oxetane, azetidine, tetrahydrofuran, dihydrofuran, dihydropyran, tetrahydropyran, etc. In some embodiments, the heterocycle is a spiro heterocycle (e.g., 2,8-diazaspiro[4.5]decane). In some embodiments, the heterocycle is a bridged heterocycle (e.g., octahydro-1H-4,7-methanoisoindole). “Spiro heterocyclyl,” or “spiro heterocycle” refers to a polycyclic heterocyclyl with rings connected through one common atom (called a spiro atom), wherein the rings have one or more heteroatoms selected from the group consisting of N, O, and S(O)ₘ (wherein m is an integer of 0 to 2) as ring atoms.

The terms “hydroxy” and “hydroxyl” as used herein refers to the radical —OH.

The term “oxo” as used herein refers to the radical ═O.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic rings may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al, Nature Reviews Drug Discovery 2008, 7, 255). For example, if a compound of the disclosure or a pharmaceutically acceptable salt, hydrate or solvate of the compound contains a carboxylic acid functional group, a prodrug can comprise an ester formed by the replacement of the hydrogen atom of the acid group with a group such as (C₁₋₈)alkyl, (C₂₋₁₂)alkylcarbonyloxymethyl, 1-(alkylcarbonyloxy)ethyl having from 4 to 9 carbon atoms, 1-methyl-1-(alkylcarbonyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino(C₂₋₃)alkyl (such as β-dimethylaminoethyl), carbamoyl-(C₁₋₂)alkyl, N,N-di(C₁-2)alkylcarbamoyl-(C₁₋₂)alkyl and piperidino-, pyrrolidino- or morpholino(C₂₋₃)alkyl.

Similarly, if a compound of the disclosure contains an alcohol functional group, a prodrug can be formed by the replacement of the hydrogen atom of the alcohol group with a group such as (C₁₋₆)alkylcarbonyloxymethyl, 1-((C₁₋₆)alkylcarbonyloxy)ethyl, 1-methyl-1-((C₁₋₆)alkylcarbonyloxy)ethyl (C₁₋₆)alkoxycarbonyloxymethyl, N—(C₁₋₆)alkoxycarbonylaminomethyl, succinoyl, (C₁₋₆)alkylcarbonyl, α-amino(C₁₋₄)alkylcarbonyl, arylalkylcarbonyl and α-aminoalkylcarbonyl, or α-aminoalkylcarbonyl-α-aminoalkylcarbonyl, where each α-aminoalkylcarbonyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

If a compound of the disclosure incorporates an amine functional group, a prodrug can be formed, for example, by creation of an amide or carbamate, an N-alkylcarbonyloxyalkyl derivative, an (oxodioxolenyl)methyl derivative, an N-Mannich base, imine or enamine. In addition, a secondary amine can be metabolically cleaved to generate a bioactive primary amine, or a tertiary amine can metabolically cleaved to generate a bioactive primary or secondary amine. For examples, see Simplicio, et al., Molecules 2008, 13, 519 and references therein.

The term “treatment” or “treating,” refers to the medical management of a patient with the intent to improve, ameliorate, stabilize (i.e., not worsen), prevent or cure a disease, pathological condition, or disorder. “Treatment” includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishing the extent of the disease or condition; preventing spread of the disease or condition; delaying or slowing the progress of the disease or condition; ameliorating or palliating the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” also includes prolonging survival as compared to expected survival if not receiving treatment. Those in need of treatment include those already with the condition or disorder, as well as those prone to have the condition or disorder or those in which the condition or disorder is to be prevented.

The term “effective amount.” “therapeutically effective amount,” or “sufficient amount” refers to a quantity sufficient to, when administered to a patient (e.g., a mammal such as a human patient), effect treatment (e.g., produce beneficial or desired results), including effects at cellular, tissue or clinical levels, etc. As such, the term depends upon the context in which it is being applied. For example, in the context of treating a disclosed neurodegenerative disease, it is an amount of a disclosed CD38 inhibitor sufficient to achieve a response as compared to the response obtained without administration of the CD38 inhibitor. The amount of a given composition described herein that will correspond to such an amount will vary depending upon various factors, such as the given CD38 inhibitor, the pharmaceutical formulation, the route of administration, the type of disease or disorder, the identity of the patient (e.g., age, sex, weight) or host being treated, and the like, but can nevertheless be routinely determined by one skilled in the art. In some embodiments, a “therapeutically effective amount” of a composition of the present disclosure is an amount that results in a beneficial or desired result in a patient (e.g., as compared to a control). A therapeutically effective amount of a composition of the present disclosure may be readily determined by one of ordinary skill by routine methods known in the art. Dosage regimen may be adjusted to provide the optimum therapeutic response.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

In some embodiments, the patient is a human. In some embodiments, the patient is an adult human patient. In some embodiments, the patient is 30 years of age or older, e.g., at least: 35, 40, 45, 50, 55, 60, 65, 70, 75, 80, 85 or 90 years old.

### I. Compounds

The disclosure is directed to, in part, to compounds that are contemplated to be modulators, e.g., inhibitors, of CD38.

For example, disclosed herein is a compound represented by Formula I:

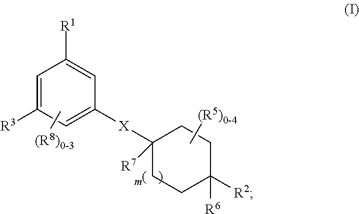

or a pharmaceutically acceptable salt and/or a stereoisomer thereof, wherein:
   X is —C(O)—NR⁴— or —S(O)₂—NR⁴—;
   R¹ is 5-6 membered monocyclic heteroaryl or 8-10 membered bicyclic heteroaryl; wherein R¹ may optionally be substituted by one or more substituents each independently selected from R¹;
   R² is selected from the group consisting of —C₁₋₆alkoxy, —C₁₋₆alkyl, halogen, hydroxyl, and hydrogen; wherein —C₁₋₆alkoxy and —C₁₋₆alkyl may optionally be substituted by one or more substituents each independently selected from R²²;
   R³ is selected from the group consisting of —C₁₋₆alkyl, —C₁₋₆alkoxy, 5-6 membered heteroaryl, 4-7 membered heterocyclyl, and halogen; wherein —C₁₋₆alkyl, —C₁₋₆alkoxy, 5-6 membered heteroaryl, and 4-7 membered heterocyclyl may optionally be substituted by one or more substituents each independently selected from R³³;
   R⁴ is selected from the group consisting of hydrogen and —C₁₋₃alkyl optionally substituted by one or more halogens;
   R⁵ and R⁸ are independently selected for each occurrence from the group consisting of hydrogen, deuterium, halogen, hydroxyl, —C₁₋₆alkyl, —C₁₋₆alkoxy, —CN, —NR^{a}R^{b}, —C(O)—NR^{a}R^{b}, and —NR^{a}—C(O)—R^{b};
   R⁶ and R⁷ are each independently selected from the group consisting of hydrogen and —C₁₋₃alkyl optionally substituted by one or more halogens;
   R¹¹, R²² and R³³ are independently selected for each occurrence from the group consisting of halogen, hydroxyl, —C₁₋₆alkyl, —C₁₋₆alkoxy, —C₃₋₆cycloalkyl, phenyl, —CN, —CF₃—NR^{a}R^{b}, —C(O)—NR^{a}R^{b}, —NR^{a}—C(O)—R^{b}, and deuterium; wherein —C₁₋₆alkyl and —C₁₋₆alkoxy may optionally be substituted by one or more substituents each independently selected from hydroxyl, halogen, and —C₁₋₃alkoxy;
   R^{a} and R^{b} are independently selected for each occurrence from the group consisting of hydrogen and —C₁₋₃alkyl optionally substituted by one or more halogens; and
   m is 0, 1 or 2.

In some embodiments, R⁵, R⁶, R⁷ and R⁸ are, for each occurrence, hydrogen. In other embodiments, a compound of the present disclosure may be represented by

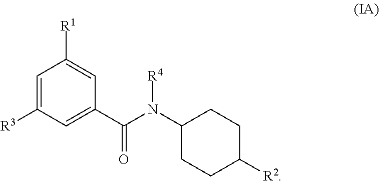

In some embodiments, R¹ is a 5-6 membered heteroaryl containing at least one ring nitrogen, wherein R¹ may optionally be substituted by one or two substituents each independently selected from R¹¹.

For example, in certain embodiments R¹ is selected from the group consisting of imidazolyl, thiazolyl, oxazolyl, pyrazolyl, triazolyl, tetrazolyl, and pyridyl, wherein R¹ may optionally be substituted by one or two substituents each independently selected from the group consisting of —CH₂OH, —OH and —NH₂.

In further embodiments, R¹ is selected from the group consisting of

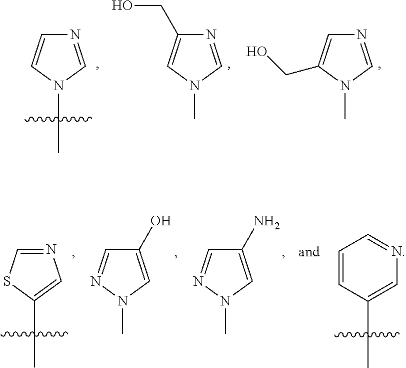

In other embodiments, R¹ is selected, for example, from the group consisting of

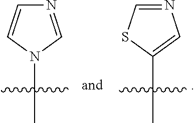 [SMILES: *n1ccnc1 ; *c1cncs1]

In some embodiments, R² is selected, for example, from the group consisting of —OCH₃, —OCH₂CH₂OCH₃, —OCH₂CH₂OCH₂CH₃, —OCH₂CH₃, —OCH₂CH₂CH₃, and —OCH(CH₃)₂. For example, in certain embodiments R² is selected from the group consisting of —OCH₃ and —OCH₂CH₂OCH₃.

In still other embodiments, R³ is, for example, —C₁₋₆alkyl. In certain embodiments, R³ is, for example, —CH₃.

In some embodiments, R³ is 5-membered heteroaryl; wherein R³ may optionally be substituted by hydroxyl or —C₁₋₃alkyl; and wherein —C₁₋₃alkyl may optionally be substituted by hydroxyl or —C₁₋₃alkyl.

For example, in certain embodiments wherein R³ is selected from the group consisting of

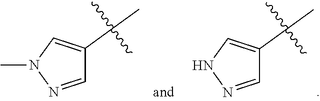

In still other embodiments, R³ is 5-6 membered heterocyclyl; wherein R³ may optionally be substituted by hydroxyl or —C₁₋₃alkyl; and wherein —C₁₋₃alkyl may optionally be substituted by hydroxyl or —C₁₋₃alkoxy.

For example, in certain embodiments R³ is selected from the group consisting of

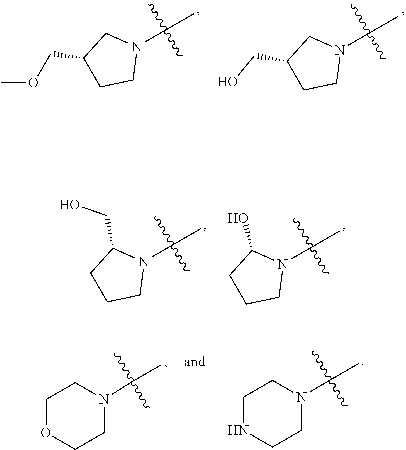

In some embodiments, R⁴ is, for example, hydrogen.

Also disclosed herein is a compound represented by Formula II:

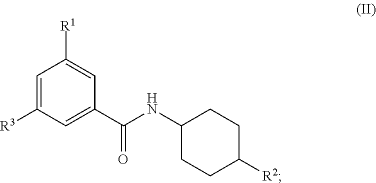

or a pharmaceutically acceptable salt and/or a stereoisomer thereof, wherein:
   R¹ is 5-6 membered heteroaryl containing at least one ring nitrogen; wherein R¹ may optionally be substituted by one, two or three substituents each independently selected from the group consisting of halogen, hydroxyl, —NH₂, —C₁₋₃alkyl, —C₁₋₃alkyl-OH, and —C₁₋₃alkoxy;
   R² is —C₁₋₆alkoxy optionally substituted by one or more substituents each independently selected from the group consisting of halogen, hydroxyl. —C₁₋₃alkyl and —C₁₋₃alkoxy; and
   R³ is selected from the group consisting of —C₁₋₆alkyl, 5-6 membered heteroaryl and 4-6 membered heterocyclyl; wherein 5-6 membered heteroaryl and 4-6 membered heterocyclyl may optionally be substituted by halogen, hydroxyl, or —C₁₋₃alkyl; and wherein —C₁₋₃alkyl and —C₁₋₃alkoxy may optionally be substituted by one or more groups selected from hydroxyl, halogen, and —C₁₋₃alkoxy.

In some embodiments, R¹ is selected, for example, from the group consisting of

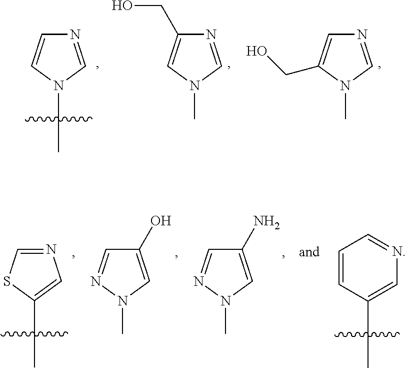

In other embodiments, R² is selected from the group consisting of —OCH₃ and —OCH₂CH₂OCH₃. In still other embodiments, R³ is selected from the group consisting of

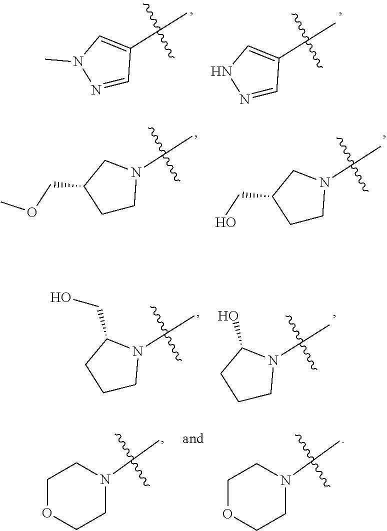

In some embodiments, the compound is a compound identified in Table 1 below or a pharmaceutically acceptable salt thereof.

TABLE 1
Exemplary compounds

| Cmpd | |
|---|---|
| No. | Structure |
| 101 | 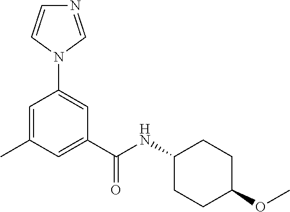 |
| 102 | 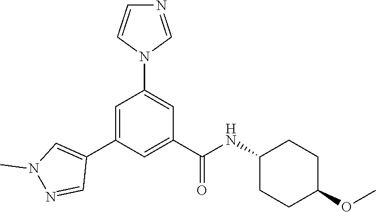 |
| 103 | 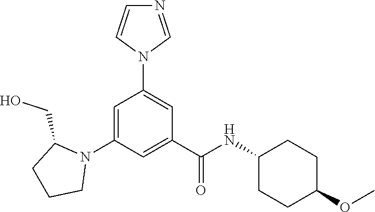 [SMILES: CO[C@H]1CC[C@H](NC(=O)c2cc(N3CCC[C@@H]3CO)cc(-n3ccnc3)c2)CC1] |
| 104 | 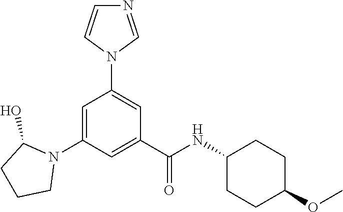 [SMILES: CO[C@H]1CC[C@H](NC(=O)c2cc(N3CCC[C@@H]3O)cc(-n3ccnc3)c2)CC1] |
| 105 | 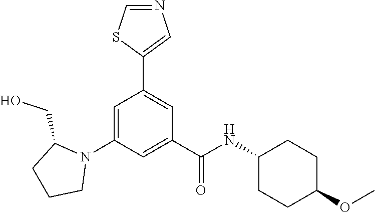 [SMILES: CO[C@H]1CC[C@H](NC(=O)c2cc(-c3cncs3)cc(N3CCC[C@@H]3CO)c2)CC1] |
| 106 | 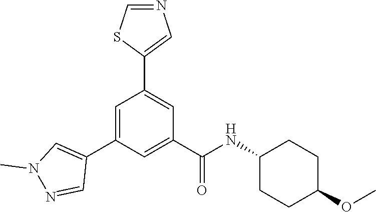 |
| 107 | 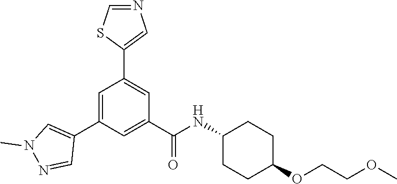 |
| 108 | 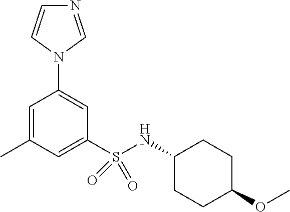 |

Procedures for making compounds described herein are provided in the examples below. In the reactions described below, it may be necessary to protect reactive functional groups (such as hydroxyl, amino, thio or carboxyl groups) to avoid their unwanted participation in the reactions. The incorporation of such groups, and the methods required to introduce and remove them are known to those skilled in the art (for example, see Greene, Wuts, Protective Groups in Organic Synthesis. 2nd Ed. (1999)). The deprotection step may be the final step in the synthesis such that the removal of protecting groups affords compounds as disclosed herein. Starting materials used in the following scheme can be purchased or prepared by methods described in the chemical literature, or by adaptations thereof, using methods known by those skilled in the art. The order in which the steps are performed can vary depending on the groups introduced and the reagents used, but would be apparent to those skilled in the art.

Compounds disclosed herein, or any of the intermediates described in the schemes above, can be further derivatised by using one or more standard synthetic methods known to those skilled in the art. Such methods can involve substitution, oxidation or reduction reactions. These methods can also be used to obtain or modify disclosed compounds or any preceding intermediates by modifying, introducing or removing appropriate functional groups.

Where it is desired to obtain a particular enantiomer of a disclosed compound, this may be produced from a corresponding mixture of enantiomers by employing any suitable conventional procedure for resolving enantiomers known to those skilled in the art. For example, diastereomeric derivatives (such as salts) can be produced by reaction of a mixture of enantiomers of a disclosed compound (such a racemate) and an appropriate chiral compound (such as a chiral base). The diastereomers can then be separated by any conventional means such as crystallization or chromatography, and the desired enantiomer recovered (such as by treatment with an acid in the instance where the diastereomer is a salt). Alternatively, a racemic mixture of esters can be resolved by kinetic hydrolysis using a variety of biocatalysts (for example, see Patel Stereoselective Biocatalysts, Marcel Decker; New York 2000).

In another resolution process a racemate of disclosed compounds can be separated using chiral High Performance Liquid Chromatography. Alternatively, a particular enantiomer can be obtained by using an appropriate chiral intermediate in one of the processes described above. Chromatography, recrystallisation and other conventional separation procedures may also be used with intermediates or final products where it is desired to obtain a particular geometric isomer of the disclosure.

In an alternative embodiment, disclosed compounds may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### II. Methods

Compounds of the present disclosure are contemplated to inhibit the activity of CD38. For example, provided herein is a method inhibiting the activity or a function of CD38 in a cell or in a patient in need of inhibition of CD38, comprising administering to the cell or patient an effective amount of a compound disclosed herein, e.g., a compound of Formula I or Formula II. Also disclosed herein, for example, is a method of treating a disease that benefits from inhibition of CD38 in a patient in need thereof, comprising administering to the patient an effective amount of a CD38 inhibitor described herein, thereby treating the disease in the patient.

Compounds of the present disclosure are contemplated to increase levels of NAD+. For example, provided herein is a method of increasing the level of NAD+ in a sample or in a patient in need thereof, comprising contacting the sample or administering to the patient an effective amount of a compound disclosed herein, e.g., a compound of Formula I or Formula II, wherein the increased level of NAD+ is relative to the level of NAD+ prior to the contacting or administering. Further disclosed herein is a method of treating a disease that benefits from an increase in NAD+ in a patient in need thereof, comprising administering to the patient an effective amount of a CD38 inhibitor described herein, e.g., a compound of Formula I or Formula II, thereby treating the disease in the patient.

Compounds of the present disclosure may be useful in the treatment of diseases associated with abnormal expression or activity of CD38. For example, the present disclosure provides a method of treating a cancer in a patient in need thereof, comprising administering to the patient an effective amount of a CD38 inhibitor described herein, e.g., a compound of Formula I or Formula II, thereby treating the cancer in the patient. In some embodiments, the cancer is characterized as having abnormal expression or activity of CD38, for example, elevated expression or activity, compared with normal cells. In other embodiments, the cancer may be selected from the group consisting of, for example, cancer of the breast, central nervous system, endometrium, kidney, large intestine, lung, esophagus, ovary, pancreas, prostate, stomach, head and neck, urinary tract, and colon. In some embodiments, the cancer is lung cancer. In other embodiments, the cancer is melanoma, In certain embodiments, the cancer is colon cancer. In still other embodiments, the cancer may be leukemia or lymphoma. Examples of lymphomas contemplated herein include, but are not limited to, Hodgkin's or non-Hodgkin's lymphoma, multiple myeloma. B-cell lymphoma (e.g., diffuse large B-cell lymphoma (DLBCL)), chronic lymphocytic lymphoma (CLL), T-cell lymphoma, hairy cell lymphoma, and Burkett's lymphoma. Examples of leukemias contemplated herein include, but are not limited to, acute lymphocytic leukemia (ALL), acute myelogenous leukemia (AML), chronic lymphocytic leukemia (CLL), and chronic myelogenous leukemia (CML). In further embodiments, the cancer may be selected from the group consisting of, for example, checkpoint-therapy treated cancers, checkpoint therapy-treated resistant cancers, adenosine-dependent tumors, Treg-infiltrated tumors, and MDSC-infiltrated tumors.

For example, contemplated cancers of the present disclosure treatable by administration of the compounds described herein may be selected from the group consisting of bladder cancer, bone cancer, glioma, breast cancer, cervical cancer, colon cancer, endometrial cancer, epithelial cancer, esophageal cancer, Ewing's sarcoma, pancreatic cancer, gallbladder cancer, gastric cancer, gastrointestinal tumors, glioma, head and neck cancer (upper aerodigestive cancer), intestinal cancers, Kaposi's sarcoma, kidney cancer, laryngeal cancer, liver cancer (e.g., hepatocellular carcinoma), lung cancer (e.g., non-small cell lung cancer, adenocarcinoma), melanoma, prostate cancer, rectal cancer, renal clear cell carcinoma, skin cancer, stomach cancer, testicular cancer, thyroid cancer, and uterine cancer. In some embodiments, contemplated cancers of the present disclosure treatable by administration of the compounds described herein may be selected from the group consisting of multiple myeloma, diffuse large B-cell lymphoma (DLBCL), hepatocellular carcinoma, bladder cancer, esophageal cancer, head and neck cancer (upper aerodigestive cancer), kidney cancer, prostate cancer, rectal cancer, stomach cancer, thyroid cancer, uterine cancer, and breast cancer.

Also provided herein are methods of treating a condition or disorder in a patient in need thereof, comprising administering to the patient an effective amount of a compound disclosed herein, e.g., a compound of Formula I or Formula II. In some embodiments, the disease or condition is selected from the group consisting of HIV/AIDS, acute lung injury, acute respiratory distress syndrome (ARDS), hyperphosphatemia, alcohol intolerance, lupus, rheumatoid arthritis ataxia-telangiectasia, sleep disorders, epilepsy, exercise intolerance, hypertension, hypoxic pulmonary vasoconstriction, hansen's disease, tuberculosis, leishmaniasis, cardiac hypertrophy, congestive heart failure (CHF), muscular dystrophy, stroke, organ reperfusion injury, idiopathic pulmonary fibrosis, pancreatitis, cystic fibrosis, asthma, chronic obstructive pulmonary disease (COPD), irritable bowel syndrome (IBS), colitis, gout, obesity, sarcopenic obesity, end stage renal disease, dyslipidemia, hearing loss, liver disease, steatosis, nonalcoholic steatohepatitis (NASH/NAFLD), Alzheimer's disease, multiple sclerosis, neurocognitive disorders, optic neuropathy, postmenopausal osteoporosis, bipolar disorder, schizophrenia, Huntington's disease, diabetes, Hartnup disease, skin hyperpigmentation, diabetic neuropathy, radiation exposure, UV skin damage, psoriasis, periodontal disease, chronic lymphocytic leukemia, amyelotrophic lateral sclerosis, Parkinson's disease, Huntington's disease, Leber's hereditary amaurosisinsulin resistance, type I diabetes, and type II diabetes.

The compounds describe herein are contemplated to have have therapeutic utility in CD38-related disorders in disease areas such as, for example, cardiology, virology, neurodegeneration, inflammation, and pain, wherein the diseases are characterized by overexpression or increased activity of CD38.

Another aspect of the disclosure provides methods of treating patients suffering from a neurodegenerative disease or disorder, e.g., Parkinson's disease. As used herein, the term “neurodegenerative disease” or “neurodegenerative disorder” encompasses a disease, disorder or condition in which cells of the central nervous system stop working or die. Neurodegenerative diseases usually get worse over time and have no cure. Such diseases may be genetic or be caused by a tumor or stroke. Neurodegenerative diseases also occur in people who consume large amounts of alcohol or are exposed to certain viruses or toxins. Non-limiting examples of neurodegenerative disorders include Parkinson's disease (PD), Alzheimer's disease (AD), amyotrophic lateral sclerosis (ALS), Huntington's disease (HD), multiple sclerosis (MS), a prion disease, spinocerebellar ataxias (SCAs), vascular dementia, frontotemporal dementia (FTD), mixed dementia, and dementia with Lewy bodies (LBD). In some embodiments, the neurodegenerative disease is a late onset disease. In other embodiments, the disclosure provides methods of treating patients suffering from neuroinflammation.

In some embodiments, the neurodegenerative disease affects basal ganglia, thalamus, red nucleus, locus coeruleus, parahippocampal gyrus, or a combination thereof. In some embodiments, the neurodegenerative disease affects basal ganglia and/or thalamus.

In some embodiments, the neurodegenerative disease is ALS, HD, PD or SCAs. In particular embodiments, the neurodegenerative disease is PD. In certain embodiments, the PD is familial Parkinson's disease.

A patient to be treated according to the methods described herein may be one who has been diagnosed with a neurodegenerative disease (e.g., PD), or one at risk of developing such a disease. Diagnosis of a neurodegenerative disease or risk of developing a neurodegenerative disease may be performed by a skilled medical professional using any suitable method or technique known in the art. One skilled in the art will understand that a patient to be treated according to the present disclosure may have been subjected to standard tests or may have been identified, without examination, as one at risk due to the presence of one or more risk factors associated with the disease or condition.

In some embodiments, the patient has a neurodegenerative disease (e.g., PD). In some embodiments, the patient has been diagnosed with a neurodegenerative disease (e.g., PD). In other embodiments, the patient is at risk of developing a neurodegenerative disease (e.g., PD). In some embodiments, the patient has or is at risk of developing PD.

For example, the disclosure provides a method of treating a neurodegenerative disease in a patient in need thereof, comprising administering to the patient an effective amount of a CD38 inhibitor described herein, e.g., a compound of Formula I or Formula II, thereby treating the neurodegenerative disease in the patient. In some embodiments, the neurodegenerative disease is, for example, Parkinson's disease, Alzheimer's disease, or Huntington's disease.

Also disclosed herein is a method of slowing the progression of Parkinson's disease in a patient in need thereof, comprising administering to the patient an effective amount of any of the compounds described herein, e.g., a compound of Formula I or Formula II, or a pharmaceutical composition comprising any of the compounds described herein, and a pharmaceutically acceptable carrier of excipient.

Also contemplated is a neurodegenerative disease that is an ocular disease or disorder comprising administering an effective amount of a disclosed CD38 inhibitor, e.g., a compound of Formula I or Formula II. For example, provided herein is a method of treating an ocular disorder such as one or more of: macular degeneration (e.g., age-related macular degeneration (AMD) or dry macular degeneration), diabetic macular edema (DME), diabetic retinopathy, glaucoma, cataracts, retinitis pigmentosa (RP), Stargardt disease, myopic macular degeneration (MMD), sub-macula hemorrhage, diabetic macular edema (DME), or uveitis.

Another aspect of the disclosure provides methods of treating patients suffering from a fatty liver disease or disorder, e.g., NAFLD or NASH. As used herein, the term “fatty liver disease” encompasses a disease, disorder or condition caused by the storage of extra fat in the liver. Alcoholic fatty liver is the accumulation of fat in the liver as a result of heavy consumption of alcohol. Nonalcoholic fatty liver disease (NAFLD) occurs in people who are not heavy alcohol consumers.

In some embodiments, the fatty liver disease is NAFLD. NAFLD is the spectrum of liver disease in which hepatic steatosis, the macrovesicular accumulation of triglyceride in hepatocytes, develops in the absence of secondary causes (e.g., medications, excessive alcohol consumption, or certain heritable conditions). In some embodiments, the NAFLD is simple hepatic steatosis (NAFL). In particular embodiments, the NAFLD is nonalcoholic steatohepatitis (NASH). NASH is the inflammatory subtype of NAFLD, with steatosis as well as evidence of hepatocyte injury (ballooning) and inflammation, with or without fibrosis.

A patient to be treated according to the methods described herein may be one who has been diagnosed with a fatty liver disease (e.g., NAFLD), or one at risk of developing such a disease. Diagnosis of a fatty liver disease or risk of developing a fatty liver disease may be performed by a skilled medical professional using any suitable method or technique known in the art. One skilled in the art will understand that a subject to be treated according to the present disclosure may have been subjected to standard tests or may have been identified, without examination, as one at risk due to the presence of one or more risk factors associated with the disease or condition.

In some embodiments, the patient has a fatty liver disease (e.g., NAFLD such as NASH). In some embodiments, the patient has been diagnosed with a fatty liver disease (e.g., NAFLD such as NASH). In other embodiments, the patient is at risk of developing a fatty liver disease (e.g., NAFLD such as NASH). In some embodiments, the patient has or is at risk of developing NAFLD. In some embodiments, the patient has or is at risk of developing NASH.

In some embodiments, the patient has NAFLD (e.g., NASH). In some embodiments, the patient also has hypertriglyceridemia, obesity, dyslipidemia, metabolic syndrome, hypertension, or type 2 diabetes, or a combination thereof.

For example, the disclosure provides a method of treating a metabolic disease in a patient in need thereof, comprising administering to the patient an effective amount of a CD38 inhibitor described herein, e.g., a compound of Formula I or Formula II, thereby treating the metabolic disease in the patient. In some embodiments, the metabolic disease is selected from the groups consisting of, for example, non-alcoholic fatty liver disease (NAFLD), non-alcoholic steatohepatitis (NASH), and diabetes (type I or type II).

In another embodiment, the disclosure provides a method of inhibiting or slowing progression of a fatty liver disease, such as NAFLD (e.g., NASH), in a patient in need thereof, comprising administering to the patient an effective amount of a compound disclosed herein, e.g., a compound of Formula I or Formula II, thereby inhibiting or slowing progression of the fatty liver disease in the patient.

In certain embodiments, the disclosure provides a method of treating the below medical indications comprising administering to a patient in need thereof a therapeutically effective amount of a compound described herein.

For example, disclosed herein is a method of treating neurodegenerative disease in patient in need thereof, comprising administering to the patient an effective amount of any of the compounds described herein, e.g., a compound of Formula I or Formula II, or a pharmaceutical composition comprising any of the compounds described herein, and a pharmaceutically acceptable carrier of excipient. In some embodiments, the neurodegenerative disease is Parkinson's disease.

Also disclosed herein is a method of treating neuroinflammation in a patient in need thereof, comprising administering to the patient an effective amount of any of the compounds described herein, e.g., a compound of Formula I or Formula II, or a pharmaceutical composition comprising any of the compounds described herein, and a pharmaceutically acceptable carrier of excipient.

Further disclosed herein is a method of treating a fatty liver disease in a patient in need thereof, comprising administering to the patient an effective amount of any of the compounds described herein, e.g., a compound of Formula I or Formula II, or a pharmaceutical composition comprising any of the compounds described herein, and a pharmaceutically acceptable carrier of excipient. In some embodiments, the fatty liver disease is nonalcoholic fatty liver disease (NAFLD). In other embodiments, the fatty liver disease is nonalcoholic steatohepatitis (NASH). In further embodiments, the the fatty liver disease is simple hepatic steatosis. In certain embodiments, treating the fatty liver disease includes slowing progression of the fatty liver disease.

In addition, disclosed herein is a method of treating fibrosis in a patient in need thereof, comprising administering to the patient an effective amount of any of the compounds described herein, e.g., a compound of Formula I or Formula II, or a pharmaceutical composition comprising any of the compounds described herein, and a pharmaceutically acceptable carrier of excipient. In some embodiments, the fibrosis is multiple organ fibrosis. In other embodiments, the fibrosis is associated with systemic sclerosis. For example, the patient suffering from fibrosis also suffers from systemic sclerosis. In still other embodiments, the fibrosis is selected from the group consisting of skin fibrosis, lung fibrosis, and peritoneal fibrosis.

In certain embodiments, a method described herein further comprises administering to the patient an additional therapeutic agent that treats a disclosed disease or disorder, or that treats a disclosed disease or disorder that is affected by, associated with, or would benefit from selective modulation, e.g., inhibition, of CD38.

Compounds described herein can be administered in combination with one or more additional therapeutic agents to treat a disorder described herein. For clarity, contemplated herein are both a fixed composition comprising a disclosed compound and another therapeutic agent such as disclosed herein, and methods of administering, separately a disclosed compound and a disclosed therapeutic. For example, provided in the present disclosure is a pharmaceutical composition comprising a compound described herein, one or more additional therapeutic agents, and a pharmaceutically acceptable excipient. In some embodiments, a disclosed compound and one additional therapeutic agent is administered. In some embodiments, a disclosed compound as defined herein and two additional therapeutic agents are administered. In some embodiments, a disclosed compound as defined herein and three additional therapeutic agents are administered. Combination therapy can be achieved by administering two or more therapeutic agents, each of which is formulated and administered separately. For example, a disclosed compound and an additional therapeutic agent can be formulated and administered separately. Combination therapy can also be achieved by administering two or more therapeutic agents in a single formulation, for example a pharmaceutical composition comprising a disclosed compound as one therapeutic agent and one or more additional therapeutic agents. For example, a disclosed compound and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other, In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X—Y—X, X—X—Y, Y—X—Y, Y—Y—X, X—X—Y—Y, etc.

In particular, in certain embodiments, the disclosure provides a method of treating the above medical indications comprising administering a patient in need thereof a therapeutically effective amount of a compound described herein, e.g., a compound of Formula I or Formula II.

### III. Pharmaceutical Compositions and Kits

Another aspect of the disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, intranasal, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions of this disclosure may be used in the form of a pharmaceutical preparation, for example, in solid, semisolid or liquid form, which contains one or more of the compound of the disclosure, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another aspect, the disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e. g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

Advantageously, the disclosure also provides kits for use by a e.g. a consumer in need of treatment of a disease or disorder described herein. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### EXAMPLES

The compounds described herein, e.g., compounds of Formula I or Formula II, can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. At least some of the compounds used as intermediates are contemplated as compounds of the disclosure.

### Example 1: Synthesis of 3-(imidazol-1-yl)-5-methyl-N-[(trans)-4-methoxycyclohexyl]benzamide (Compound 101)

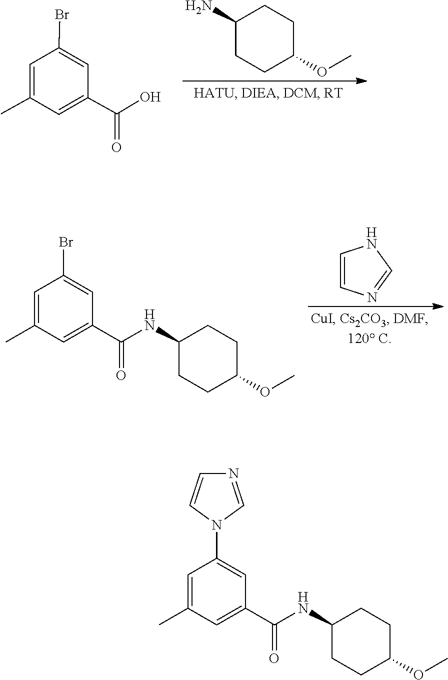

To a mixture of 3-bromo-5-methylbenzoic acid (1.0 g, 4.650 mmol, 1 equiv) and trans-4-methoxycyclohexan-1-amine (600 mg, 4.650 mmol, 1 equiv) in DCM (200 mL) were added HATU (2.65 g, 6.975 mmol, 1.5 equiv) and DIEA (1.8 g, 13.950 mmol, 3 equiv) in portions at 0° C. under air atmosphere. The resulting mixture was stirred for additional 1 h at room temperature. The resulting mixture was concentrated under reduced pressure. The residue was purified by reverse phase flash chromatography with the following conditions: column, C18 silica gel; mobile phase, MeCN in water (0.1% formic acid), 10% to 100% gradient in 25 min; detector, UV 254 nm. Desired product could be detected by LCMS. This resulted in 3-bromo-5-methyl-N-[(trans)-4-methoxycyclohexyl]benzamide (490 mg, 32.30% yield) as a yellow solid.

A solution of 3-bromo-5-methyl-N-[(trans)-4-methoxycyclohexyl]benzamide (400 mg, 1.226 mmol, 1 equiv), imidazole (125 mg, 1.839 mmol, 1.5 equiv), K₂CO₃ (508 mg, 3.678 mmol, 3 equiv) and CuI (467 mg, 2.452 mmol, 2 equiv) in DMF (8 mL) was stirred for 1 h at 120° C. under nitrogen atmosphere. The resulting mixture was filtered; the filter cake was washed with MeOH (3×8 mL). The filtrate was concentrated under reduced pressure. The residue was purified by reverse phase flash chromatography with the following conditions: column, C18 silica gel; mobile phase, MeCN in water (0.1% formic acid), 10% to 100% gradient in 25 min; detector, UV 254 nm. Desired product could be detected by LCMS. This resulted in 3-(imidazol-1-yl)-5-methyl-N-[(trans)-4-methoxvcyclohexyl]benzamide (52.3 mg, 13.53%) as a white solid. ¹H NMR (400 MHz, DMSO-d₆) δ 8.27 (d, J=7.9 Hz, 2H), 7.80 (d, J=15.9 Hz, 2H), 7.64 (s, 2H), 7.13 (s, 1H), 3.81-3.72 (m, 1H), 3.25 (s, 3H), 3.18-3.08 (m, 1H), 2.42 (s, 3H), 2.11-1.98 (m, 2H), 1.98-1.62 (m, 2H), 1.52-1.27 (m, 2H), 1.27-1.16 (m, 2H). LCMS (ESI) [M+H]⁺: 314.10.

### Example 2: Synthesis of 3-[(3S)-3-hydroxypyrrolidin-1-yl]-5-(imidazol-1-yl)-N-[(trans)-4-methoxycyclohexyl]benzamide (Compound 104)

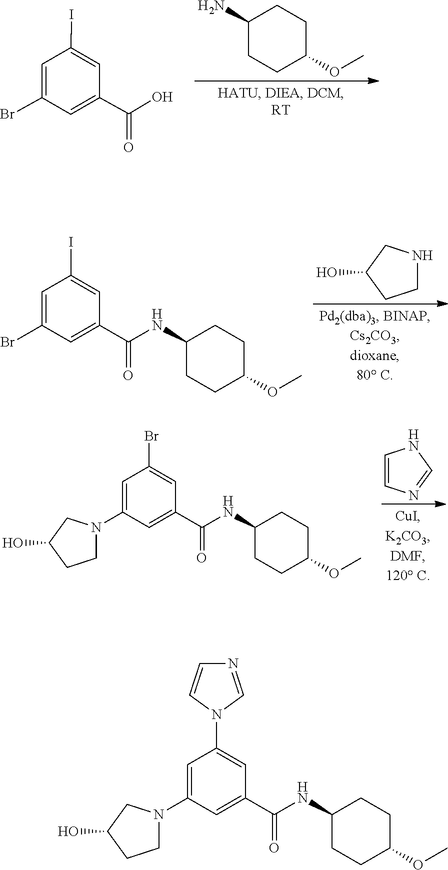

To a stirred mixture of 3-bromo-5-iodobenzoic acid (500 mg, 1.529 mmol, 1 equiv) and trans-4-methoxycyclohexan-1-amine (296 mg, 2.293 mmol, 1.5 equiv) in DCM (15 mL) were added DIEA (988 mg, 7.645 mmol, 5 equiv) and HATU (1.16 g, 3.058 mmol, 2 equiv) in portions at room temperature. The resulting mixture was stirred for additional 2 h at room temperature. The resulting mixture was concentrated under vacuum. The residue was purified by reverse phase flash chromatography with the following conditions: column, C18 silica gel; mobile phase, MeCN in water (0.1% formic acid), 0% to 100% gradient in 25 min; detector, UV 254 nm. This resulted in 3-bromo-5-iodo-N-[(trans)-4-methoxycyclohexyl]benzamide (325 mg, 48.55% yield) as a yellow oil.

To a stirred mixture of 3-bromo-5-iodo-N-[(trans)-4-methoxycyclohexyl]benzamide (300 mg, 0.685 mmol, 1 equiv) and (3S)-pyrrolidin-3-ol (89 mg, 1.028 mmol, 1.5 equiv) in dioxane (4 mL) were added Pd₂(dba)₃ (63 mg, 0.069 mmol, 0.1 equiv), Cs₂CO₃ (669 mg, 2.055 mmol, 3 equiv) and BINAP (85 mg, 0.137 mmol, 0.2 equiv) in portions at room temperature under nitrogen atmosphere. The resulting mixture was stirred for additional 12 h at 80° C. The resulting mixture was concentrated under vacuum. The residue was purified by reverse phase flash chromatograph y with the following conditions: column, C18 silica gel; mobile phase, MeCN in water (0.1% formic acid), 0% to 100% gradient in 25 min; detector, UV 254 nm. This resulted in 3-bromo-5-[(3S)-3-hydroxypyrrolidin-1-yl]-N-[(trans)-4-methoxycyclohexyl]benzamide (133 mg, 49.0 1% yield) as a brown oil.

To a mixture of 3-bromo-5-[(3S)-3-hydroxypyrrolidin-1-yl]-N-[(trans)-4-methoxycyc lohexyl]benzamide (160 mg, 0.403 mmol, 1 equiv) and imidazole (137 mg, 2.015 mmol, 5 equiv) in DMF (5 mL) were added K₂CO₃ (166.97 mg, 1.209 mmol, 3 equiv) and CuI (153.39 mg, 0.806 mmol, 2 equiv) in portions at room temperature under nitrogen atmosphere. The resulting mixture was stirred for additional 12 h at 120° C. The resulting mixture was filtered; the filter cake was washed with MeOH (3×10 mL). The filtrate was concentrated under reduced pressure. The residue was purified by reverse phase flash chromatography with the following conditions: column, C18 silica gel; mobile phase, MeCN in water (0.1% formic acid), 0% to 100% gradient in 25 min; detector, UV 254 nm. This resulted in 3-[(3S)-3-hydroxypyrrolidin-1-yl]-5-(imidazol-1-yl)-N-[(trans)-4-methoxycyclohexyl]benzamide (30.23 mg, 19.52% yield) as a white solid. ¹H NMR (400 MHz, DMSO-d₆) δ 8.25 (s, 1H), 8.15 (d, J=7.8 Hz, 1H), 7.75 (s, 1H), 7.18 (s, 1H), 7.10 (s, 1H), 6.90 (s, 1H), 6.77 (s, 1H), 5.01 (s, 1H), 4.43 (s, 1H), 3.91-3.62 (m, 1H), 3.53-3.39 (m, 3H), 3.25 (s, 3H), 3.25-3.18 (m, 1H), 3.16-3.07 (m, 1H), 2.11-1.98 (m, 3H), 1.98-1.76 (m, 3H), 1.53-1.30 (m, 2H), 1.30-1.09 (m, 2H). LCMS (ESI) [M+H]⁺: 385.15.

### Example 3: Synthesis of 3-[(3S)-3-(hydroxymethyl)pyrrolidin-1-yl]-5-(imidazol-1-yl)-N-[(trans)-4-methoxycyclohexyl]benzamide (Compound 103)

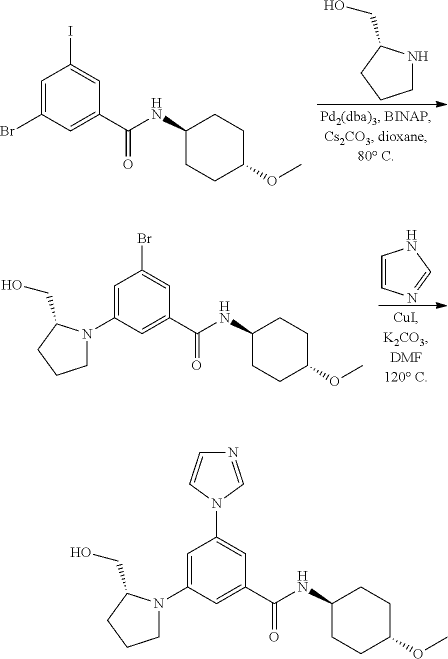 [SMILES: CO[C@H]1CC[C@H](NC(=O)c2cc(Br)cc(I)c2)CC1 ; OC[C@H]1CCCN1 ; CO[C@H]1CC[C@H](NC(=O)c2cc(Br)cc(N3CCC[C@@H]3CO)c2)CC1 ; c1c[nH]cn1 ; CO[C@H]1CC[C@H](NC(=O)c2cc(N3CCC[C@@H]3CO)cc(-n3ccnc3)c2)CC1]

To a mixture of 3-bromo-5-iodo-N-[(trans)-4-methoxycyclohexyl]benzamide (300 mg, 0.685 mmol, 1 equiv) and (3S)-pyrrolidin-3-ylmethanol (83 mg, 0.822 mmol, 1.2 equiv) in dioxane (5 mL) were added Pd₂(dba)₃ (63 mg, 0.069 mmol, 0.1 equiv), Cs₂CO₃ (669 mg, 2.055 mmol, 3 equiv) and BINAP (85 mg, 0.137 mmol, 0.2 equiv) in portions at room temperature under nitrogen atmosphere. The resulting mixture was stirred for additional 12 h at 80° C. The resulting mixture was concentrated under vacuum. The residue was purified by reverse phase flash chromatography with the following conditions: column, C18 silica gel; mobile phase, MeCN in water (0.1% formic acid), 0% to 100% gradient in 25 min; detector, UV 254 nm. This resulted in 3-bromo-5-[(3S)-3-(hydroxymethyl)pyrrolidin-1-yl]-N-[(trans)-4-methoxycyclohexyl]benzamide (223.16 mg, 79.23% yield) as a brown oil.

To a mixture of 3-bromo-5-[(3S)-3-(hydroxymethyl)pyrrolidin-1-yl]-N-[(trans)-4-metho xycyclohexyl]benzamide (220 mg, 0.535 mmol, 1 equiv) and imidazole (182 mg, 2.675 mmol, 5 equiv) in DMF (5 mL) were added K₂CO₃ (222 mg, 1.605 mmol, 3 equiv) and CuI (204 mg, 1.070 mmol, 2 equiv) in portions at room temperature under nitrogen atmosphere. The resulting mixture was stirred for additional 12 h at 120° C. The resulting mixture was filtered; the filter cake was washed with MeOH (3×10 mL). The filtrate was concentrated under reduced pressure. The residue was purified by reverse phase flash chromatography with the following conditions: column, C18 silica gel; mobile phase, MeCN in water (0.1% formic acid), 10% to 100% gradient in 25 min; detector, UV 254 nm. This resulted in 3-[(3S)-3-(hydroxymethyl)pyrrolidin-1-yl]-5-(imidazol-1-yl)-N-[(trans)-4-methoxycyclohexyl]benzamide (28.35 mg, 13.30% yield) as a white solid. ¹HNMR (400 MHz, DMSO-d₆) δ 8.21 (s, 1H), 8.13 (d, J=7.8 Hz, 1H), 7.71 (s, 1H), 7.18 (s, 1H), 7.10 (s, 1H), 6.99 (s, 1H), 6.84 (s, 1H), 4.83 (s, 1H), 3.93-3.68 (m, 2H), 3.58-3.42 (m, 2H), 3.24 (s, 3H), 3.22-3.18 (m, 1H), 3.17-2.90 (m, 2H), 2.12-2.00 (m, 4H), 1.99-1.81 (m, 4H), 1.52-1.30 (m, 2H), 1.28-1.11 (m, 2H). LCMS (ESI) [M+H]⁺: 399.10.

### Example 4: Synthesis of -(imidazol-1-yl)-5-(1-methylpyrazol-4-yl)-N-[(trans)-4-methoxycyclohexyl]benzamide (Compound 102)

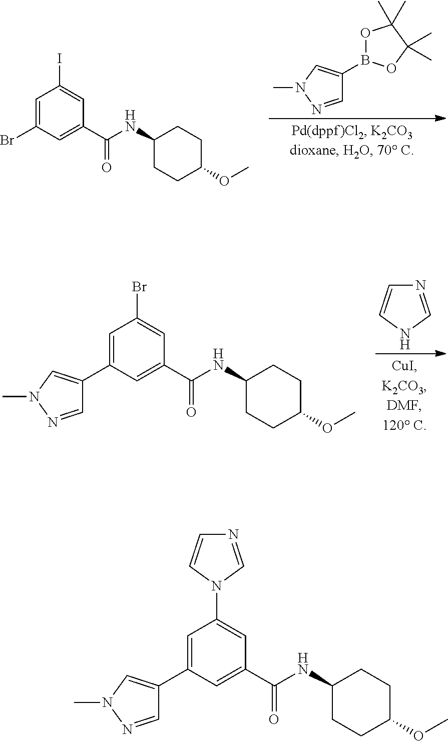

A mixture of 3-bromo-5-iodo-N-[(trans)-4-methoxycyclohexyl]benzamide (500 mg, 1.141 mmol, 1 equiv) and 1-methyl-4-(4,4,5,5-tetramethyl-1,3,2-dioxaborolan-2-yl)pyrazole (356 mg, 1.712 mmol, 1.5 equiv), K₂CO₃ (473 mg, 3.423 mmol, 3 equiv) and Pd(dppf)Cl₂ (93 mg, 0.114 mmol, 0.1 equiv) in dioxane (8 mL) and H₂O (2 mL) was stirred for 1 h at 70° C. under nitrogen atmosphere. The resulting mixture was concentrated under vacuum. The residue was purified by reverse phase flash chromatography with the following conditions: column, C18 silica gel; mobile phase, MeCN in water (0.1% formic acid), 10% to 100% gradient in 25 min; detector, UV 254 nm to afford 3-bromo-5-(1-methylpyrazol-4-yl)-N-[(trans)-4-methoxycyclohexyl]benzamide (159 mg, 35.59% yield) as a brown oil.

To a mixture of 3-bromo-5-(1-methylpyrazol-4-yl)-N-[(trans)-4-methoxycyclohexyl]benzamide (110 mg, 0.280 mmol, 1 equiv) and imidazole (19 mg, 0.280 mmol, 1 equiv) in DMF (5 mL) were added CuI (107 mg, 0.560 mmol, 2 equiv) and K₂CO₃ (116 mg, 0.840 mmol, 3 equiv) in portions at room atmosphere. The resulting mixture was stirred for 16 h at 120° C. under nitrogen atmosphere. The resulting mixture was filtered; the filter cake was washed with MeOH (3×50 mL). The filtrate was concentrated under reduced pressure. The residue was purified by reverse phase flash chromatography with the following conditions: column, C18 silica gel; mobile phase, MeCN in water (0.1% formic acid), 10% to 100% gradient in 20 min; detector, UV 254 nm. This resulted in 3-(imidazol-1-yl)-5-(1-methylpyrazol-4-yl)-N-[(trans)-4-methoxycyclohexyl]benzamide (46.35 mg, 43.56% yield) as a white solid. ¹H NMR (400 MHz, DMSO-d₆) δ 8.52 (s, 1H), 8.32 (d, J=5.7 Hz, 2H), 8.07-7.81 (m, 5H), 7.25 (s, 1H), 3.90 (s, 3H), 3.86-3.74 (m, 1H), 3.26 (s, 3H), 3.14 (s, 1H), 2.13-2.00 (m, 2H), 1.92 (d, J=12.5 Hz, 2H), 1.52-1.38 (m, 2H), 1.29-1.12 (m, 2H). LCMS (ESI) [M+H]⁺: 380.20.

### Example 5: Synthesis of 3-(1-methylpyrazol-4-yl)-N-[(trans)-4-(2-methoxyethoxy) cyclohexyl]-5-(1,3-thiazol-5-yl)benzamide (Compound 107)

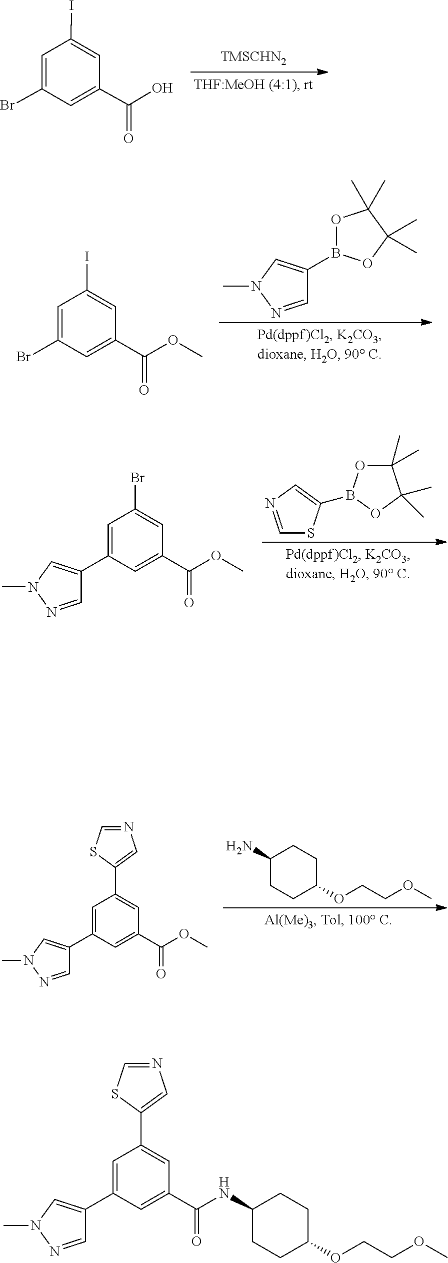

A solution of 3-bromo-5-iodobenzoic acid (1 g, 3.059 mmol, 1 equiv) and TMSCHN₂ (699 mg, 6.118 mmol, 2 equiv) in THF (6 mL) and MeOH (1.5 mL) was stirred for 1 h at room temperature. The resulting mixture was concentrated under reduced pressure. The residue was purified by reverse phase flash chromatography with the following conditions: column, C18 silica gel; mobile phase, MeCN in water (0.1% formic acid), 10% to 50% gradient in 10 min; detector, UV 254 nm. This resulted in methyl 3-bromo-5-iodobenzoate (500 mg, 47.94% yield) as a white solid.

A mixture of methyl 3-bromo-5-iodobenzoate (500 mg, 1.467 mmol, 1 equiv), 1-methyl-4-(4,4,5,5-tetramethyl-1.3.2-dioxaborolan-2-yl)pyrazole (457.71 mg, 2.200 mmol, 1.5 equiv), Pd(dppf)Cl₂ (107 mg, 0.147 mmol, 0.1 equiv) and K₂CO₃ (608 mg, 4.401 mmol, 3 equiv) in dioxane (6 mL) and H₂O (1.5 mL) was stirred for 1 h at 90° C. under nitrogen atmosphere. The resulting mixture was concentrated under reduced pressure. The residue was purified by reverse phase flash chromatography with the following conditions: column, C18 silica gel; mobile phase, MeCN in water (0.1% formic acid), 10% to 50% gradient in 10 min; detector, UV 254 nm. This resulted in methyl 3-bromo-5-(1-methylpyrazol-4-yl)benzoate (150 mg, 34.66% yield) as a white solid.

A solution of methyl 3-bromo-5-(1-methylpyrazol-4-yl)benzoate (500 mg, 1.694 mmol, 1 equiv), Pd(dppf)Cl₂ (123.96 mg, 0.169 mmol, 0.1 equiv), 5-(4,4,5,5-tetramethyl-1,3,2-dioxaborolan-2-yl)-1,3-thiazole (715.23 mg, 3.388 mmol, 2 equiv) and K₂CO₃ (702.41 mg, 5.082 mmol, 3 equiv) in H₂O (0.5 mL) and dioxane (2 mL) was stirred for 1 h at 90° C. under nitrogen atmosphere. The resulting mixture was concentrated under reduced pressure. The residue was purified by reverse phase flash chromatography with the following conditions: column, C18 silica gel; mobile phase, MeCN in water (0.1% formic acid), 10% to 50% gradient in 10 min; detector, UV 254 nm. This resulted in methyl 3-(1-methylpyrazol-4-yl)-5-(1,3-thiazol-5-yl)benzoate (150 mg, 29.58% yield) as a white solid.

A solution of methyl 3-(1-methylpyrazol-4-yl)-5-(1,3-thiazol-5-yl)benzoate (150 mg, 0.501 mmol, 1 equiv), trans-4-(2-methoxyethoxy)cyclohexan-1-amine (87 mg, 0.501 mmol, 1 equiv) and Al(Me)₃ (72 mg, 1.002 mmol, 2 equiv) in toluene (0.5 mL) was stirred for 1 h at 100° C. under nitrogen atmosphere. The reaction was quenched with sat. NH₄Cl (aq.) at room temperature. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by reverse phase flash chromatography with the following conditions: column, C18 silica gel; mobile phase, MeCN in water (0.1% formic acid), 10% to 50% gradient in 10 min; detector, UV 254 nm. This resulted in 3-(1-methylpyrazol-4-yl)-N-[(trans)-4-(2-methoxyethoxy) cyclohexyl]-5-(1,3-thiazol-5-yl)benzamide (20.6 mg, 9.33% yield) as a white solid. ¹H NMR (400 MHz, DMSO-d₆) δ 9.15 (s, 1H), 8.46 (s, 1H), 8.33 (d, J=9.9 Hz, 2H), 8.03 (d, J=2.5 Hz, 2H), 7.96 (s, 1H), 7.85 (s, 1H), 3.90 (s, 3H), 3.83-3.75 (m, 1H), 3.58-3.51 (m, 2H), 3.43 (d, J=5.1 Hz, 3H), 3.25 (s, 3H), 2.09-2.00 (m, 2H), 1.98-1.88 (m, 2H), 1.49-1.34 (m, 2H), 1.30-1.20 (m, 2H). LCMS (ESI) [M+H]⁺: 441.10.

### Example 6: Synthesis of 3-[(2R)-2-(hydroxymethyl)pyrrolidin-1-yl]-N-[(trans)-4-methoxycyclohexyl]-5-(1,3-thiazol-5-yl)benzamide (Compound 105)

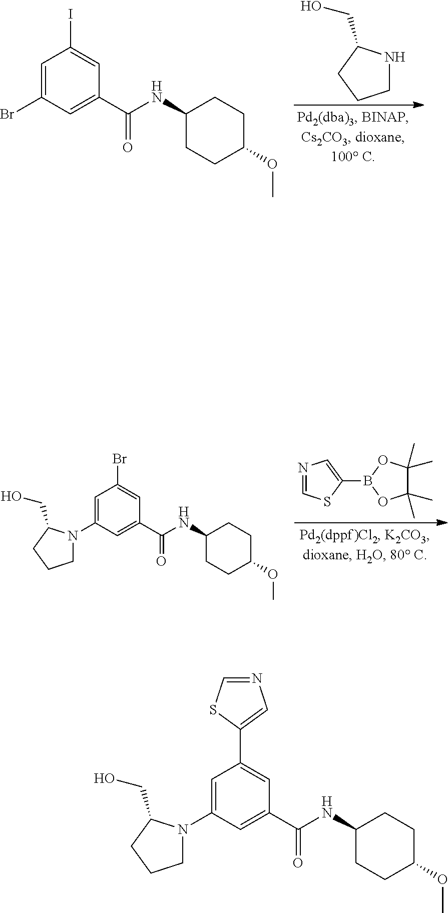 [SMILES: CO[C@H]1CC[C@H](NC(=O)c2cc(Br)cc(I)c2)CC1 ; OC[C@H]1CCCN1 ; CO[C@H]1CC[C@H](NC(=O)c2cc(Br)cc(N3CCC[C@@H]3CO)c2)CC1 ; CC1(C)OB(c2cncs2)OC1(C)C ; CO[C@H]1CC[C@H](NC(=O)c2cc(-c3cncs3)cc(N3CCC[C@@H]3CO)c2)CC1]

A mixture of 3-bromo-5-iodo-N-[(trans)-4-methoxycyclohexyl]benzamide (200 mg, 0.46 mmol, 1 equiv), prolinol (69 mg, 0.69 mmol, 1.5 equiv), Pd₂(dba)₃ (4 mg, 0.05 mmol, 0.1 equiv), BINAP (57 mg, 0.09 mmol, 0.2 equiv) and Cs₂CO₃ (446 mg, 1.37 mmol, 3 equiv) in dioxane (5 mL) was stirred overnight at 100° C. under nitrogen atmosphere. The resulting mixture was concentrated under reduced pressure. The crude product was purified by reverse phase flash chromatography with the following conditions (column. C18 silica gel; mobile phase, ACN in water (0.1% formic acid), 5% to 100% gradient in 30 min; detector, UV 254 nm and UV 220 nm) to afford 3-bromo-5-[(2R)-2-(hydroxymethyl)pyrrolidin-1-yl]-N-[(trans)-4-methoxycyclohexyl]benzamide (100 mg, 53.25% yield) as a white solid.

A mixture of 3-bromo-5-[(2R)-2-(hydroxymethyl) pyrrolidin-1-yl]-N-[(trans)-4-methoxy cyclohexyl]benzamide (95 mg, 0.23 mmol, 1 equiv), 5-(4,4,5,5-tetramethyl-1,3,2-dioxaborolan-2-yl)-1,3-thiazole (73 mg, 0.35 mmol, 1.5 equiv), Pd(dppf)Cl₂ (17 mg, 0.02 mmol, 0.1 equiv) and K₂CO₃ (96 mg, 0.69 mmol, 3 equiv) in dioxane (4 mL) and H₂O (0.4 mL) was stirred overnight at 80° C. under nitrogen atmosphere. The resulting mixture was concentrated under reduced pressure. The crude product was purified by reverse phase flash chromatography with the following conditions (column, C18 silica gel; mobile phase, ACN in water (0.1% formic acid), 5% to 100% gradient in 30 min; detector, UV 254 nm and UV 220 nm) to afford 3-[(2R)-2-(hydroxymethyl)pyrrolidin-1-yl]-N-[(trans)-4-methoxycyclohexyl]-5-(1,3-thiazol-5-yl) benzamide (15.9 mg, 16.10% yield) as a white solid. LCMS (ESI) [M+H]⁺: 416.20. ¹H NMR (400 MHz, DMSO-d₆) δ 9.08 (s, 1H), 8.30 (s, 1H), 8.19 (d, J=7.9 Hz, 1H), 7.27 (d, J=1.5 Hz, 1H), 7.00 (s, 1H), 6.94 (s, 1H), 4.91-4.78 (m, 1H), 3.83-3.71 (m, 2H), 3.54-3.46 (m, 2H), 3.25 (s, 4H), 3.17-3.08 (m, 2H), 2.08-1.86 (m, 8H), 1.42-1.31 (m, 2H), 1.28-1.18 (m, 2H).

### Example 7: Synthesis of 3-(1-methylpyrazol-4-yl)-N-[(trans)-4-methoxycyclohexyl]-5-(1,3-thiazol-5-yl)benzamide (Compound 106)

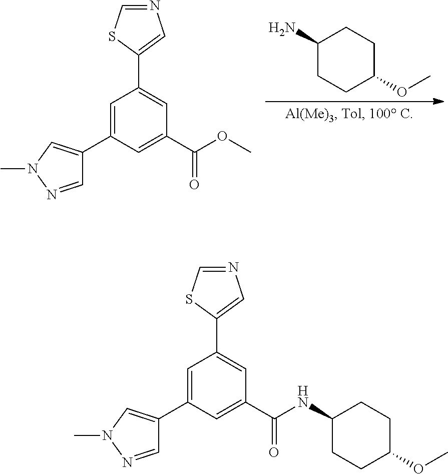

To a stirred mixture of methyl 3-(1-methylpyrazol-4-yl)-5-(1,3-thiazol-5-yl)benzoate (120 mg, 0.401 mmol, 1 equiv) and (trans)-4-methoxycyclohexan-1-amine (62 mg, 0.481 mmol, 1.2 equiv) in toluene (5 mL) was added Al(Me)₃ (86 mg, 1.203 mmol, 3 equiv) at 0° C. The resulting mixture was stirred for 1 h at

100° C. under nitrogen atmosphere. The reaction was quenched with sat. NH₄Cl (aq.) at room temperature. The resulting mixture was extracted with EtOAc (3×30 mL). The combined organic layers were washed with brine, dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% formic acid), 10% to 100% gradient in 25 min; detector, UV 254 nm to afford 3-(1-methylpyrazol-4-yl)-N-[(trans)-4-methoxycyclohexyl]-5-(1,3-thiazol-5-yl)benzamide (20.3 mg, 12.77% yield) as a white solid. LCMS (ESI) [M+H]⁺:397.15. ¹H NMR (400 MHz, DMSO-d₆) δ 9.14 (s, 1H), 8.45 (s, 1H), 8.37-8.29 (m, 2H), 8.03 (d, J=1.8 Hz, 2H), 7.96 (s, 1H), 7.85 (s, 1H), 3.90 (s, 3H), 3.85-3.76 (m, 1H), 3.25 (s, 3H), 3.17-3.10 (m, 1H), 2.09-2.01 (m, 2H), 1.96-1.88 (m, 2H), 1.42-1.34 (m, 2H), 1.14-1.25 (m, 2H).

### Example 8: Synthesis of Compound 3-bromo-5-methyl-N-[(trans)-4-methoxycyclohexyl]benzenesulfonamide (Compound 108)

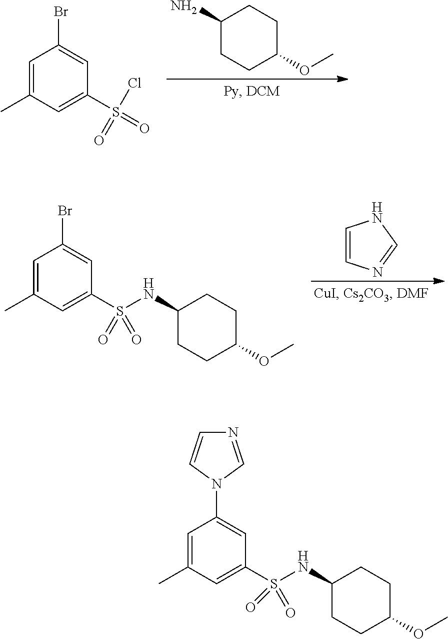

A solution of (trans)-4-methoxycyclohexan-1-amine (254 mg, 1.972 mmol, 1.00 equiv) in DCM (10 mL) and pyridine (10 mL) was added 3-bromo-5-methylbenzenesulfinyl chloride (500 mg, 1.972 mmol, 1 equiv) in three portions at 0° C. The resulting mixture was stirred overnight at room temperature. The mixture was diluted with water (100 mL). The resulting mixture was extracted with DCM (3×100 mL), dried over anhydrous Na₂SO₄. After filtration, the filtrate was concentrated under reduced pressure. The residue was purified by reverse phase flash chromatography with the following conditions: column, C18 silica gel; mobile phase, MeCN in water (0.1% formic acid), 10% to 50% gradient in 10 min; detector, UV 254 nm. This resulted in 3-bromo-5-methyl-N-[(trans)-4-methoxycyclohexyl]benzenesulfonamide (240 mg, 33.59% yield) as a light yellow oil.

A mixture of 3-bromo-5-methyl-N-[(trans)-4-methoxycyclohexyl]benzenesulfonamide (500 mg, 1.380 mmol, 1 equiv), imidazole (140 mg, 2.070 mmol, 1.50 equiv), Cs₂CO₃ (1.34 g, 4.140 mmol, 3.00 equiv) and CuI (525 mg, 2.760 mmol, 2.00 equiv) in DMF (6 mL) was stirred overnight at 120° C. under nitrogen atmosphere. The resulting mixture was filtered; the filter cake was washed with MeOH (3×10 mL). The filtrate was concentrated under reduced pressure. The residue was purified by reverse phase flash chromatography with the following conditions: column, C18 silica gel; mobile phase, MeCN in water (0.1% formic acid), 10% to 100% gradient in 30 min; detector. UV 254 nm. This resulted in 3-(imidazol-1-yl)-5-methyl-N-[(trans)-4-methoxycyclohexyl]benzene sulfonamide (34.5 mg, 7.05% yield) as a light yellow solid. LCMS (ESI) [M+H]⁺: 350.15. ¹H NMR (300 MHz, DMSO-d₆) δ 9.15-7.90 (m, 1H) 7.89-7.79 (m, 2H), 7.75-7.60 (m, 2H), 7.58-6.80 (m, 1H), 3.34 (s, 3H), 3.10-2.94 (m, 2H), 2.47 (s, 3H), 1.98-1.75 (m, 2H), 1.67-1.45 (m, 2H), 1.31-0.86 (m, 4H).

### Example 9: CD38 Hydrolase Inhibition Assay

The CD38 hydrolase inhibition assay measures the glycohydrolase activity of CD38, using an NAD analog as substrate and measuring formation of a fluorescent ADP ribose analog. A 4× buffer was prepared with 1M sucrose and 160 mM Tris-HCl at pH 7.4, for a final assay concentration of 250 mM sucrose and 40 mM Tris-HCl. The 4× solution was kept at 4° C. and warmed to room temperature before use. Tween20 was added to 1× buffer for a final concentration of 0.05%. Human recombinant CD38 (BPS Bioscience) was diluted to 0.4 ng/μL in 1× buffer and 12.5 μL added to each plate well. Inhibitors were added and DMSO content was normalized to the highest concentration on the plate, with an upper limit of 0.5%. Finally, ε-NAD was diluted to 200 μM in 1× buffer and 12.5 μL added to each plate well, for final well concentrations of 0.2 ng/μL CD38 and 100 μM ε-NAD. Plates were covered and incubated at room temperature for 15 m, and then read on a plate reader using excitation 300 nm and emission 410 nm. 200 nM 78c was included as a fully inhibited well, as well as DMSO-only wells as uninhibited. IC50s for test compounds were determined either using GraphPad Prism software v9.3, or CDD Vault. The results are shown in Table 2.

TABLE 2

| Cmpd No. | IC50 (uM) |
|---|---|
| 101 | ++ |
| 102 | + |
| 103 | ++ |
| 104 | ++ |
| 105 | ++ |
| 107 | + |
| 108 | +++ |

+ <0.1;
++ 0.1 to 1.0;
+++ >1.0

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A compound represented by Formula I: 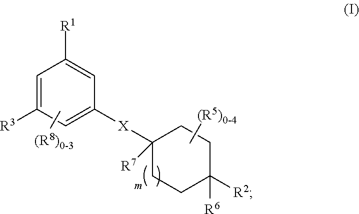
or a pharmaceutically acceptable salt and/or a stereoisomer thereof, wherein:
X is —C(O)—NR⁴— or —S(O)₂—NR⁴—;
R¹ is 5-6 membered monocyclic heteroaryl or 8-10 membered bicyclic heteroaryl; wherein R¹ may optionally be substituted by one or more substituents each independently selected from R¹¹;
R² is selected from the group consisting of —C₁₋₆alkoxy, —C₁₋₆alkyl, halogen, hydroxyl, and hydrogen; wherein —C₁₋₆alkoxy and —C₁₋₆alkyl may optionally be substituted by one or more substituents each independently selected from R²²;
R³ is selected from the group consisting of —C₁₋₆alkyl, —C₁₋₆alkoxy, 5-6 membered heteroaryl, 4-7 membered heterocyclyl, and halogen; wherein —C₁₋₆alkyl, —C₁₋₆alkoxy, 5-6 membered heteroaryl, and 4-7 membered heterocyclyl may optionally be substituted by one or more substituents each independently selected from R³³;
R⁴ is selected from the group consisting of hydrogen and —C₁₋₃alkyl optionally substituted by one or more halogens;
R⁵ and R⁸ are independently selected for each occurrence from the group consisting of hydrogen, deuterium, halogen, hydroxyl, —C₁₋₆alkyl, —C₁₋₆alkoxy, —CN, —NR^{a}R^{b}, —C(O)—NR^{a}R^{b}, and —NR^{a}—C(O)—R^{b};
R⁶ and R⁷ are each independently selected from the group consisting of hydrogen and —C₁₋₃alkyl optionally substituted by one or more halogens;
R¹¹, R²² and R³³ are independently selected for each occurrence from the group consisting of halogen, hydroxyl, —C₁₋₆alkyl, —C₁₋₆alkoxy, —C₃₋₆cycloalkyl, phenyl, —CN, —CF₃—NR^{a}R^{b}, —C(O)—NR^{a}R^{b}, —NR^{a}—C(O)—R^{b}, and deuterium; wherein —C₁₋₆alkyl and —C₁₋₆alkoxy may optionally be substituted by one or more substituents each independently selected from hydroxyl, halogen, and —C₁₋₃alkoxy;
R^{a} and R^{b} are independently selected for each occurrence from the group consisting of hydrogen and —C₁₋₃alkyl optionally substituted by one or more halogens; and
m is 0, 1 or 2.

2. The compound of claim 1, wherein R⁵, R⁶, R⁷ and R⁸ are, for each occurrence, hydrogen.

3. The compound of claim 1 or 2, wherein the compound is represented by

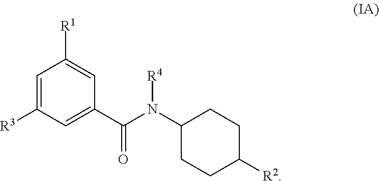

4. The compound of any one of claims 1-3, wherein R¹ is a 5-6 membered heteroaryl containing at least one ring nitrogen, wherein R¹ may optionally be substituted by one or two substituents each independently selected from R¹.

5. The compound of any one of claims 1-4, wherein R¹ is selected from the group consisting of imidazolyl, thiazolyl, oxazolyl, pyrazolyl, triazolyl, tetrazolyl, and pyridyl, wherein R¹ may optionally be substituted by one or two substituents each independently selected from the group consisting of —CH₂OH, —OH and —NH₂.

6. The compound of any one of claims 1-5, wherein R¹ is selected from the group consisting of

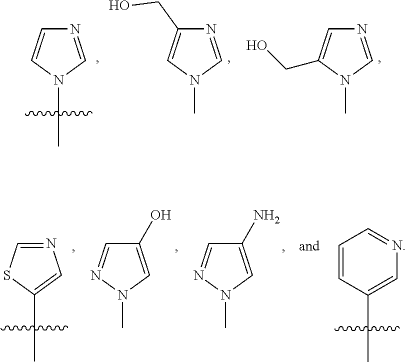

7. The compound of any one of claims 1-6, wherein R¹ is selected from the group consisting of

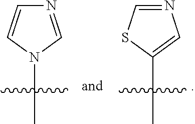 [SMILES: *n1ccnc1 ; *c1cncs1]

8. The compound of any one of claims 1-7, wherein R² is selected from the group consisting of —OCH₃, —OCH₂CH₂OCH₃, —OCH₂CH₂OCH₂CH₃, —OCH₂CH₃, —OCH₂CH₂CH₃, and —OCH(CH₃)₂.

9. The compound of any one of claims 1-8, wherein R² is selected from the group consisting of —OCH₃ and —OCH₂CH₂OCH₃.

10. The compound of any one of claims 1-9, wherein R³ is —C₁₋₆alkyl.

11. The compound of any one of claims 1-10, wherein R³ is —CH₃.

12. The compound of any one of claims 1-8, wherein R³ is 5-membered heteroaryl; wherein R³ may optionally be substituted by hydroxyl or —C₁₋₃alkyl; and wherein —C₁₋₃alkyl may optionally be substituted by hydroxyl or —C₁₋₃alkyl.

13. The compound of any one of claims 1-8 and 12, wherein R³ is selected from the group consisting of

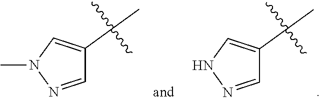

14. The compound of any one of claims 1-8, wherein R³ is 5-6 membered heterocyclyl; wherein R³ may optionally be substituted by hydroxyl or —C₁₋₃alkyl; and wherein —C₁₋₃alkyl may optionally be substituted by hydroxyl or —C₁₋₃alkoxy.

15. The compound of any one of claims 1-8 and 14, wherein R³ is selected from the group consisting of

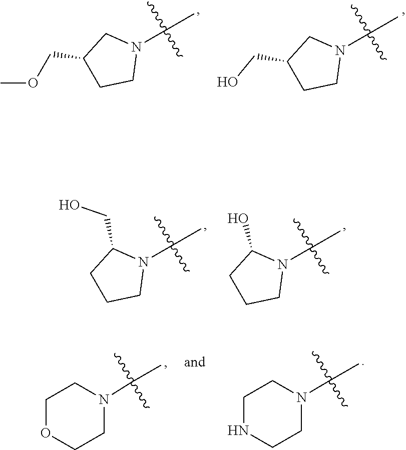

16. The compound of any one of claims 1-12, wherein R⁴ is hydrogen.

17. A compound represented by Formula II: 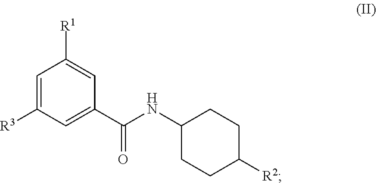
or a pharmaceutically acceptable salt and/or a stereoisomer thereof, wherein:
R¹ is 5-6 membered heteroaryl containing at least one ring nitrogen; wherein R¹ may optionally be substituted by one, two or three substituents each independently selected from the group consisting of halogen, hydroxyl, —NH₂, —C₁₋₃alkyl, —C₁₋₃alkyl-OH, and —C₁₋₃alkoxy;
R² is —C₁₋₆alkoxy optionally substituted by one or more substituents each independently selected from the group consisting of halogen, hydroxyl, —C₁₋₃alkyl and —C₁₋₃alkoxy; and
R³ is selected from the group consisting of —C₁₋₆alkyl, 5-6 membered heteroaryl and 4-6 membered heterocyclyl; wherein 5-6 membered heteroaryl and 4-6 membered heterocyclyl may optionally be substituted by halogen, hydroxyl, or —C₁₋₃alkyl; and wherein —C₁₋₃alkyl and —C₁₋₃alkoxy may optionally be substituted by one or more groups selected from hydroxyl, halogen, and —C₁₋₃alkoxy.

18. The compound of claim 17, wherein R¹ is selected from the group consisting of

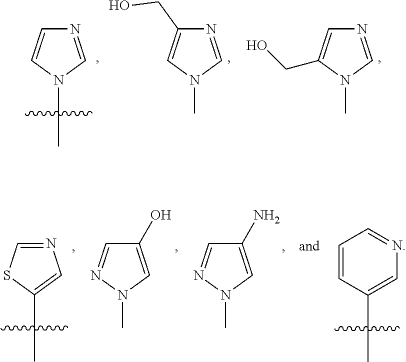

19. The compound of claim 17 or 18, wherein R² is selected from the group consisting of —OCH₃ and —OCH₂CH₂OCH₃.

20. The compound of any one of claims 17-18, wherein R³ is selected from the group consisting of

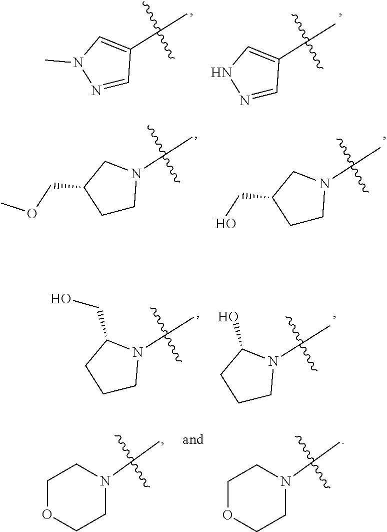

21. A compound selected from the group consisting of: 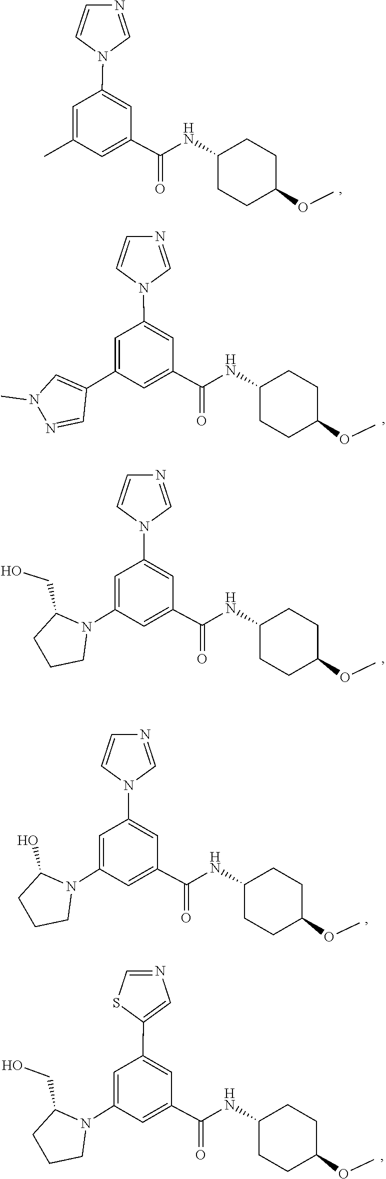 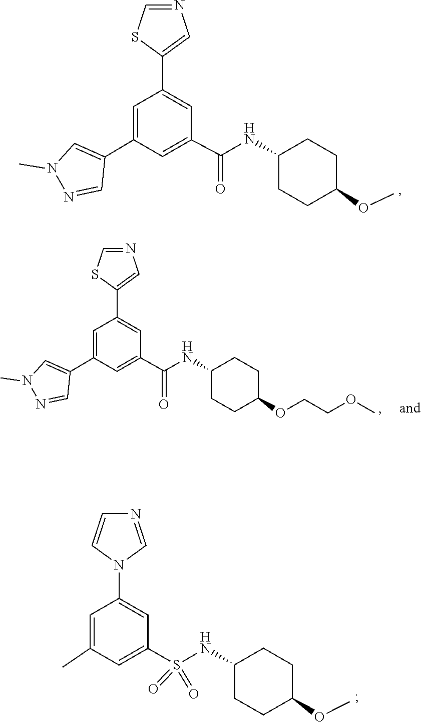
or a pharmaceutically acceptable salt and/or a stereoisomer thereof.

22. A pharmaceutical composition comprising the compound of any one of claims 1-21, and a pharmaceutically acceptable excipient.

23. A method of treating a disease that benefits from inhibition of CD38 in a patient in need thereof, comprising administering to the patient an effective amount of a a compound of any one of claims 1-21, or the pharmaceutical composition of claim 22.

24. A method of treating a disease that benefits from an increase in NAD+ in a patient in need thereof, comprising administering to the patient an effective amount of a compound of any one of claims 1-21, or the pharmaceutical composition of claim 22.

25. A method of treating neurodegenerative disease in a patient in need thereof, comprising administering to the patient an effective amount of a compound of any one of claims 1-21, or the pharmaceutical composition of claim 22.

26. The method of claim 25, wherein the neurodegenerative disease is Parkinson's disease.

27. A method of treating a cancer in a patient in need thereof, comprising administering to the patient an effective amount of a compound of any one of claims 1-21, or the pharmaceutical composition of claim 22.

28. The method of claim 27, wherein the cancer is selected from the group consisting of lung cancer, breast cancer, melanoma, and colon cancer.

29. A method of treating a patient suffering from fibrosis, and in need thereof, comprising administering to the patient an effective amount of a compound of any one of claims 1-21, or the pharmaceutical composition of claim 22.

30. The method of claim 30, wherein the patient suffering from fibrosis also suffers from systemic sclerosis.

31. The method of claim 30 or 31, wherein the fibrosis is selected from the group consisting of skin fibrosis, lung fibrosis, and peritoneal fibrosis.

32. A method of treating a fatty liver disease in a patient in need thereof, comprising administering to the patient an effective amount of a compound of any one of claims 1-21, or the pharmaceutical composition of claim 22.

33. The method of claim 29, wherein the fatty liver disease is selected from the group consisting of nonalcoholic fatty liver disease (NAFLD) and nonalcoholic steatohepatitis (NASH).
